(19) United States
(12) Patent Application Publication
(10) Pub. No.: US 2003/0068298 A1
(43) Pub. Date: Apr. 10, 2003

(54) Method for treatment of intracerebral tumors

(76) Inventor: Svadovskiy Aleksandr Igorevich, Moscow (RU)

Correspondence Address: SMITH & HOPEN PA, 15950 BAY VISTA DRIVE, SUITE 220, CLEARWATER, FL, 33760

(21) Appl. No.: 10/065,311

(22) Filed: Oct. 2, 2002

(30) Foreign Application Priority Data
Oct. 9, 2001 (RU) 2001127259

(51) Int. Cl. A61K 38/20

(52) U.S. Cl. 424/85.2; 424/93.7

(57) ABSTRACT

The method for treatment of intracerebral tumors, consists in performing cytokine immunotherapy followed by neurosurgical intervention for a total resection of the intracerebral tumor. The cytokine therapy comprises intravenous administration of a preset dose of lymphokine-activated killer cells together with recombinant yeast interleukin-2 in a daily dose of up to 2 million IU, the administration being in fact a prolonged infusion from one to three days for four or five hours daily.

### BACKGROUND OF INVENTION

1. Cross Reference to Related Disclosures

This application claims priority from Russian Application No. 2001127259, filed Oct. 9, 2001, the specification of which is incorporated herein by reference.

2. Field of Invention

The present invention relates in general to medicine and has particular reference to a method for treatment of intracerebral tumors.

The invention can find application in the various fields of medicine, e.g., in neurosurgery, neuro-oncology, or neuroimmunology.

3. Background of Invention

Intracerebral tumors are liable to relapse, the recurrence rate depending both on the grade of anaplasia and of the proliferative potential, and on the scope of surgical intervention (i.e., total or subtotal tumor resection).

A theory of “tumorigenic field” (W. Willis, 1953) is heretofore known, according to which there are multiple focal proliferates establishing a tumorigenic field. The tumor grows from the center towards the periphery until all foci merge into a single tumor nodule; once the “tumorigenic field” has been spent completely, the tumor grows further “on its own”. After a neurosurgical intervention for resection of the tumor nodule there may be left a zone consisting of separate tumor cells which are not detectable by objective visualization using computer-aided and magnetic-resonance tomography on account of a limited resolution of instruments and apparatus. It is such survived tumor cells that are responsible for recurrence of the diseases.

A method for treatment of intracerebral tumors (cf. RU Patent No. 2,133,624 C1) is known to consist in that there is effected cytokine immunotherapy followed by neurosurgical intervention for total resection of a intracerebral tumor. Cytokine immunotherapy includes intravenous administration (as prolonged infusions) of a predetermined dose of recombinant yeast interleukin-2.

When administering pure recombinant yeast interleukin-2, despite positive changes in patient's immunodeficiency (both quantitative and qualitative indices), the presence of a tumor in patient's organism provides for producing and suppressing the effectors of a cell-level component of immune system due to production (by the tumor tissue) of immune response blocking substances (such as growth transforming factor β₁ and β₂, prostaglandins, and some of interleukins).

Administration of pure interleukin-2 produces a systemic effect on immunodeficient cells, i.e., stimulates all kinds of lymphocytes, but no emphasize is placed upon maintaining the most important, from the viewpoint of destroying tumor tissue, the CD16+ lymphocyte population. It is just said population that is able to interact immediately, without antigens of Class I-II of the Major Hystocompatibility Complex, with a tumor cell in order to annihilate it. Hence no adequately rapid induced immunocytokine reaction occurs in patient's vascular system, nor there is provided a necessary effect of activated lymphocytes directly on the tumor tissue and perifocal zone. Furthermore, the tumor fails to soften adequately enough to perform an easier mechanical (viz., less traumatogenic radical resection thereof. Lethal postoperative misadventures and pronounced side effects are possible. Patient's postoperative life span, according to the method under discussion, is increased but inconsiderably.

### DETAILED DESCRIPTION

Therefore the present invention has for its principal object to provide a method for treatment of intracerebral tumors, said method providing, due to a more rapid induced immunocytokine reaction in patient's vascular system, an effect of activated lymphocytes directly on the tumor tissue and perifocal zone, thereby destroying or softening the tumor and rendering it less dense so as to perform a lighter mechanical, viz., less traumatogenic radical tumor resection, which eventually allows of adding to patient's postoperative life span and prevents the onset of lethal misadventures or pronounced side-effects.

The aforesaid object is accomplished due to the fact that in a method for treatment of intracerebral tumors, said method comprising cytokine immunotherapy followed by neurosurgical intervention consisting in a total resection of the intracerebral tumor, according to the invention, said cytokine therapy comprises intravenous administration of a preset dose of lymphokine-activated killer cells together with recombinant yeast interleukin-2 in a daily dose of up to 2 million IU, said administration appearing as prolonged infusion (from one to three days for four or five hours daily). It is expedient that said cytokine immunotherapy be preceded either by open or stereotaxis biopsy of a space-occupying brain structure.

It is also reasonable that two or three weeks after said neurosurgical intervention, a course of cytokine immunotherapy be conducted, comprising intravenous administration of a preset dose of lymphokine-activated killer cells together with recombinant yeast interleukin-2 in a daily dose of up to 2 million IU, said administration appearing as prolonged infusion (from one to three days for four or five hours daily).

The herein-proposed method for treatment of intracerebral tumors allows, due to a faster induced immunocytokine reaction in the patient's vascular system, providing the effect of activated lymphocytes directly on the tumor tissue and perifocal zone, thereby destroying or softening the tumor and rendering it less dense, which makes possible an easier mechanical radical resection, thus eventually allows of adding to patient's postoperative life span and prevents the onset of lethal misadventures or pronounced side-effects. With the present method of treatment use of a systemic intravenous administration is more physiologically appropriate from the viewpoint of providing an induced immune attack upon the bed of the excised tumor through arterial blood, and liquidating immunodeficiency of blood. Moreover, the present method of treatment makes it possible to dispense with the use of a catheter in the space previously occupied by the removed tumor and of the Ommaya reservoir. Unlike other heretofore-known treatment methods which are capable of treating only Grade III or IV intracerebral tumors (gliomas), the method in question allows of treating gliomas of all grades, that is I, II, III, and IV according to WHO classification. The present invention also make it possible to dispense with traditional chemo- and/or radiotherapy following neurosurgical intervention.

The proposed method allows of facilitating surgical ablation of neoplastic tissue, rendering surgical intervention less traumatic, as well as of extending the recurrence-free period (adjuvant immunotherapy) in case of intracerebral tumors.

In what follows the invention is illustrated by specific examples of its practical realization.

The proposed method for treatment of intracerebral tumors consists in that there is effected cytokine immunotherapy followed by neurosurgical intervention for total resection of a intracerebral tumor. Cytokine immunotherapy includes intravenous administration, in the form of prolonged infusion within from one to three days for four or five hours daily, a preset dose of lymphokine-activated killer cells together with recombinant yeast interleukin-2 in a daily dose of up to 2 million IU.

For practicing therapy with lymphokine-activated killer cells (LAK therapy) the following procedures are carried out: withdrawing 800 ml patient's venous blood; spinning-down lymphocytes; adding a known sterile natural medium to lymphocytes sediment; incubating the thus-prepared lymphocyte suspension for 72 hours at 37° C.; and sedimenting the cells in a centrifuge. When preparing said lymphocyte suspension, the culture medium may be doped with sterile interleukin-1 and interleukin-2 (Roncoleukin) solutions. Then the thus-prepared lymphocyte suspension is added to a flask filled with 400 ml physiological saline preheated to 37° C. The flask contents together with interleukin-2 (International Pharmaco-EEG Group “Night Biennial IPEG Meeting” Abstracts Prague, Sep. 12-14, 1996, p.21, “Analysis of the brain electrical activity before . . . ”, V. V. Gnezdicsky, A. I. Svadovsky et al.) is administered to the patient by prolonged infusion.

The present recombinant yeast interleukin-2 is less toxic. The preset dose thereof depends on a number clinical reasons, that is, patient's age, tumor size, and degree of immunodeficiency. It is expedient that cytokine immunotherapy be preceded open or stereotaxic biopsy of the space-occupying brain structure (that is, intracerebral tumor).

The pathogenetic reason for carrying out therapy of intracerebral tumor patients is reduced indices of peripheral blood cellular immunity, low degree of tumor tissue lymphocytic infiltration, as well as established ability of activated lymphocytes and other cytokines to penetrate the blood-brain barrier (BBB) in order to lyse the tumor cells.

To stimulate transforming mature peripheral blood lymphocytes into lymphokine-activated killer cells, use is made of home-produced recombinant interleukin-2 prepared with the use of a yeast culture and free from toxic effects.

Recombinant yeast interleukin-2 may be used not only as a transformation inducer of LAK cells but also as a background preparation prior to practicing LAK therapy with a view to enhance patient's cellular immunity. It is likewise expedient that recombinant yeast interleukin-2 be used following neurosurgical intervention and LAK therapy for preventive maintenance of the cellular link of immunity.

Known from current literature are techniques for introducing lymphokine-activated killer (LAK) cells in both the tumor itself and in the bed of resected tumor through the Ommaya reservoir (cf. Jacobs S. K. et al., 1986; Ingram M., et al., 1990; Yoshida S. Et al., 1992). In the present treating method use is made of systemic intravenous administration of LAK cells, thus providing a prolonged immune attack upon the tumor cells to maintain a definite concentration of killer cells in the blood bed, and to liquidate immunodeficiency. The method proposed herein allows of dispensing with Ommanya reservoir, thus avoiding superimposition of secondary infection.

It is due to the herein-proposed method for treating intracerebral tumors by introducing in the blood bed the patient's lymphokine-activated killer cells themselves which are capable of penetrating the blood-brain barrier and getting in direct contact with the tumor cells that lytic changes in the tumor occur, thus rendering resection of the tumor nodule less traumatic. Moreover, the proposed method makes it possible to affect in the postoperative period some individual tumor cells disposed along the periphery of the resected tumor nodule, thereby retarding the rate of tumor recurrence or ruling out any recurrence whatever. Growing lymphokine-activated lymphocyte killer cells CD16+ outside of the organism and their reintroducing into the organism's blood bed renders said killer cells more competitive with respect to other cells in the organism which have not been withdrawn. Accordingly, said killer cells possess higher killing potential against tumor cells.

It is also reasonable that the neurosurgical intervention be followed (two or three weeks later) by a course of cytokine immunotherapy comprising intravenous administration (in the form of prolonged infusion, within one to three days for four or five hours daily) of a preset dose of lymphokine-activated killer cells together with recombinant yeast interleukin-2, said preset dose amounting to 2 million IU daily.

Interleukin-2 and LAK cells to a considerable extent add to the penetrance of the BLOOD-BRAIN BARRIER (possessing selective penetrance) and enable high-molecular medicaments (e.g., interferons) to penetrate the tumor tissue, thus ensuring partial or complete tumor destruction. It is found that the proposed LAK therapy promotes peritumoral edema so that the clearance of transudate occurs via the unilateral portion of the brain ventricular system and the subarachnoid space according to the universal mechanism in the same way as in case of extensive local brain injuries in the form of brain contusion or intracerebral hematomas (cf. Svadovsky A. I., Potapov A. A., Likhterman L. B. et al., CT and MRI Evaluation of Traumatic Brain Edema and Its Biochemical and Histological Correlates/In Book: Intracranial Pressure VIII.-Springer-Verlag. Berlin-Heidelberg-New-York. 1993, pp.499-502).

An analysis of tumor biopsy specimens using light and electron microscopes and data obtained from typing of lymphocytes in tumor biopsy specimens involving use of monoclonal antibodies give evidence of an ability of activated lymphocytes to lyse gliomas cells in patients with both low and high grade of anaplasia, which substantiates preoperative treatment in case of low-malignancy gliomas.

Use of the present treatment method in the preoperative period facilitates extirpation of tumor tissue and renders operative intervention less traumatic. Using radionuclide gamma-encephalography it has been established ability of the proposed method to enhance penetrance of the blood-brain barrier only directly in the parenchyma of an intracerebral tumor but never in an intact brain. In our observations of radionuclide gamma-encephalography we have pointed out the focus of a cytokine-induced inflammation appearing as a ring (or semi-ring). This provides a basis for preventive (adjuvant) use of the proposed therapy following a radical surgical intervention aimed at further struggle against the survived tumor cells disposed around the bed of the resected tumor.

The present method for treatment of intracerebral tumors has been tested clinically. Clinical trials were carried out under conditions of the neurological and neurosurgical clinic “Neuroaesculap” in Moscow. The method was applied for treating patients with intracerebral tumors, in particular, in astrocytomas of the mixed, fibrillary, protoplasmic, and anaplastic types, as well as in glioblastomas and gliosarcomas, oligoastrocytomas, medulloblastomas, and some other affections.

Given below are specific exemplary case histories illustrating the present method for neurosurgical treatment of intracerebral tumors.

### EXAMPLE 1

Male patient B., 48 has been admitted to the clinic with complaints of headache, tottering gate, affected eyesight. The diagnosis made at another stationary medical institution was one of intracerebral tumor of the right frontal lobe of the brain. Astrocytoma of the I-II grade of anaplasia. In 1997 there was performed surgery for subtotal resection of the tumor. The surgical intervention was followed by a course of radiotherapy with a radiation dose of 40 Gy. In view of an aggravated patient's state a magnetic-resonance imaging study was performed. Further tumor growth, brain descensional dislocation to the left, and perifocal edema were detected. The patient was given cytokine immunotherapy in the form of intravenous administration of a preset dose of lymphokine-activated killer cells together with recombinant yeast interleukin-2 up to a daily dose of 2 million IU, said administration being in the form of a prolonged infusion (within three days for four hours daily). In a week after completing said cytokine immunotherapy, osteoplastic trepanation of the skull and tumor extirpation was performed. During surgery the tumor appeared as a considerably decomposed mass, especially in the central portion thereof. The postoperative period was uneventful. Headache regressed. Tottering gate was corrected. Eyesight recovered. The operative wound healed by first intention. The follow-up period lasted for 2.5 years.

### EXAMPLE 2

Male patient G., 33. The diagnosis was one of the Third degree anaplastic astrocytoma. In 1995 osteoplastic trepanation of the skull and resection of the tumor in the right front lobe of the brain were carried out at another clinic. In 1998 the patient was admitted to the clinic with recurrence of anaplastic astrocytoma of the right front lobe. The patient was given preoperative immunotherapy comprising intravenous administration of a preset dose of lymphokine-activated killer cells together with recombinant yeast interleukin-2 up to a daily dose of 2 million IU, said administration being in the form of a prolonged infusion (within one to three days for four or five hours daily).

The patient was given surgical intervention for osteoplastic trepanation of the skull and resection of the partially destroyed tumor of the right frontal lobe. The operative wound healed by first intention. Three weeks after surgery the patient was given a repeated course of cytokine immunotherapy which comprised intravenous administration of a preset dose of lymphokine-activated killer cells together with recombinant yeast interleukin-2 up to a daily dose of 2 million IU, said administration being in the form of a prolonged infusion (within three days for four hours daily).

No relapses were observed. The follow-up period lasted for 4 years.

### EXAMPLE 3

Female patient S., 27. The diagnosis was one of intracerebral tumor of the fronto-parieto-temporal lobes of the brain. Open biopsy was performed in order to confirm a neoplastic nature of the space-occupying brain structure. It was established that said structure is in fact an intracerebral tumor, i.e., astrocytoma of the I grade anaplasia. The patient was given cytokine immunotherapy.

Cytokine therapy involved intravenous administration of a preset dose of lymphokine-activated killer cells together with recombinant yeast interleukin-2 up to a daily dose of 2 million IU, said administration being in the form of a prolonged infusion (for five hours within one day). Next a neurosurgical intervention was performed for subtotal tumor resection. Control data gave evidence of absence of any signs of tumor recurrence for a period of a few months. The follow-up period lasted for 5 years.

### EXAMPLE 4

Male patient Sh., 57. The diagnosis was one of glioblastoma of the left parieto-occipital region. While in the clinic the patient was given cytokine immunotherapy which included intravenous administration of a preset dose of lymphokine-activated killer cells together with recombinant yeast interleukin-2 up to a daily dose of 2 million IU, said administration being in the form of a prolonged infusion (for four hours daily within one day). Thereupon the patient was subjected to osteoplastic trepanation of the skull and tumor resection. Three weeks after the surgery the patient was given a course of cytokine immunotherapy involving intravenous administration of a preset dose of lymphokine-activated killer cells together with recombinant yeast interleukin-2 up to a daily dose of 2 million IU, said administration being in the form of a prolonged infusion (within three days for four hours daily) with a view to preventing further tumor growth. The follow-up period lasted for 5 years.

### EXAMPLE 5

Male patient Kh., 48. Has been admitted to the clinic with complaints of stubborn headache of a diffuse nature and convulsive seizures. In the spring of 1993 the patient sustained stereotaxic biopsy of a space-occupying process in the ventricular triangle of the left cerebral hemisphere. Histological conclusion: astrocytoma of the II grade anaplasia.

Thereafter the patient refused of operative intervention in favor of domestic treatment. However, in view of aggravated state (higher rate of convulsive seizures, loss of working capacity) the patient was admitted to the clinic for examination and treatment in June of 1997, where he was suggested a course of immunotherapy according to the herein-proposed method which was carried out by intravenous administration of a preset dose of lymphokine-activated killer cells together with recombinant yeast interleukin-2 up to a daily dose of 2 million IU, said administration being in the form of a prolonged infusion (within three days for four hours daily). Next the patient was subjected to osteoplastic trepanation of the skull in the left parieto-temporal region of the brain and total resection of the intracerebral tumor in the lateral divisions of the region of the brain ventricular triangle. Histological conclusion: oligoastrocytoma of the II-III grades of anaplasia. The operative wound healed by first intention. The follow-up period lasts up till now.

### EXAMPLE 6

Male patient G., 33. Has been admitted to the clinic with complaints of convulsive seizures (three events all in all). The patient was examined at the diagnostic center where computerized tomography of the patient's brain detected a space-occupying process in the right frontal lobe of the brain, said process playing the part of a mass-effect that compresses the unilateral brain ventricular system. Magnetic resonance tomography corroborated the presence of a space-occupying structure. With a view to more accurately defining the diagnosis and as a first stage of treatment, open biopsy of said space-occupying structure in the right frontal lobe of the brain was performed early in June, 1997. Histological conclusion was one of a tumor of the astrocytic series. The patient was suggested a course of cytokine immunotherapy involving intravenous administration of a preset dose of lymphokine-activated killer cells together with recombinant yeast interleukin-2 up to a daily dose of 2 million IU, said administration being in the form of a prolonged infusion (for four hours within one day). On Jun. 17, 1997 was performed osteoplastic trepanation of the skull for total resection of the intracerebral tumor of the right frontal lobe. Histological conclusion: anaplastic astrocytoma. No postoperative complications were observed. The follow-up period lasted for 5 years.

Hence clinical trials of the proposed method for treatment of intracerebral tumors enables one to infer that said method is capable of providing, due to a more rapid induced immunocytokine reaction in patient's vascular system, an effect of activated lymphocytes directly on the tumor tissue and perifocal zone, thereby destroying or softening the tumor and rendering it less dense so as to perform an easier mechanical, viz., less traumatogenic radical tumor resection, which eventually allows of adding to patient's postoperative life span and prevents the onset of lethal misadventures or pronounced side-effects.

1. A method for treatment of intracerebral tumors, in performing cytokine immunotherapy followed by neurosurgical intervention for a total resection of the intracerebral tumor, wherein said cytokine therapy comprises intravenous administration of a preset dose of lymphokine-activated killer cells together with recombinant yeast interleukin-2 in a daily dose of up to 2 million Iu, said administration appearing as prolonged infusion:

2. The method of claim 1 wherein the prolonged infusion period is substantially between one to three days.

3. The method of claim 2 wherein the period of daily infusion is approximately four to five hours per day.

4. The method for treatment of intracerebral tumors as claimed in claim 1, wherein said cytokine immunotherapy is preceded either by open or stereotaxis biopsy of a space-occupying brain structure.

5. The method for treatment of intracerebral tumors as claimed in claim 1, wherein two or three weeks after said neurosurgical intervention, a course of cytokine immunotherapy is conducted, comprising intravenous administration of a preset dose of lymphokine-activated killer cells together with recombinant yeast interleukin-2 in a daily dose of up to 2 million IU, said administration appearing as prolonged infusion from one to three days for four or five hours daily.

6. The method for treatment of intracerebral tumors as claimed in claim 4, wherein two or three weeks after said neurosurgical intervention, a course of cytokine immunotherapy is conducted, comprising intravenous administration of a preset dose of lymphokine-activated killer cells together with recombinant yeast interleukin-2 in a daily dose of up to 2 million IU, said administration appearing as prolonged infusion from one to three days for four or five hours daily.
